(19) United States
(12) Patent Application Publication
(10) Pub. No.: US 2004/0249458 A1
(43) Pub. Date: Dec. 9, 2004

(54) Slotted ball or disc and nail porter securement stapedial prosthesis and instrument for implantation of said prosthesis in a stapedectomy procedure

(76) Inventors: Shay Kahana, D.N. Jordan Valley (IL); Yeshaya Yarnitsky, Haifa (IL); Noder Bartal, D.N.Jordan Valley (IL)

Correspondence Address: SHAY KAHANA, KIBBUTS BET-ZERA, D.N. JORDAN VALLEY, 15135, IL

(21) Appl. No.: 10/859,132

(22) Filed: Jun. 3, 2004

Related U.S. Application Data
(60) Provisional application No. 60/475,479, filed on Jun. 4, 2003.

(51) Int. Cl. A61F 2/18; A61F 11/00

(52) U.S. Cl. 623/10

(57) ABSTRACT

An improved stapedial prosthesis of the piston type replaces the stapes of the middle ear comprising a slotted ball or disc for receiving the lenticular process of the incus and a nail porter which closes and opens the nail porter without causing damage to the incus and an instrument that is used to implant the prosthetic piston in the stapedectomy procedure.

This application claims the benefit of prior U.S. application No. 60/475,479, filed Jun. 4, 2003 and is incorporated herein by reference.

### ABSTRACT

A slotted ball or disc and nail porter securement stapedial piston prosthesis and an instrument for holding the prosthesis in stapedectomy procedure comprises a handle, a tube attached to the handle for holding the prosthesis, and a rod inside the tube from the handle to the free end of the tube for releasing the prosthesis from the instrument. The handle connects to a vacuum source for securing the prosthesis which prevents the rotation and tilting of the prosthesis in an axis parallel to the prosthesis at the opening end of the tube.

### REFERENCES CITED

### U.S. Patent Documents

U.S. Pat. No. 3,196,462 July 1965 Robinson

U.S. Pat. No. 3,711,869 January 1973 Shea

U.S. Pat. No. 3,899,822 August 1975 Beverly W.Armstrong

U.S. Pat. No. 3,931,648 January 1976 Shea, Jr.3/1

U.S. Pat. No. 4,292,693 Oct. 6, 1981 Shea & Griggs.

U.S. Pat. No. 5,171,240 Dec. 15, 1992 Yuthaphong

U.S. Pat. No. 5,935,167 Aug. 10, 1999 Wengen

U.S. Pat. No. 6,432,139 Aug. 13, 2002 Elies & Carsten

### Other Publications

Catalog, Micro-Surgery Instruments and Implants,

Richards Manufacturing Co., 1965.

### 1. FIELD OF THE INVENTION

The invention relates to a new and improved medical stapedial prosthesis and an instrument for use in implanting a prosthetic piston in the middle ear in a stapedectomy procedure, more particularly to an improved stapedial prosthesis and a method for implanting the stapedial prosthesis.

### 2. BACKGROUND OF THE INVENTION

The middle ear comprises the malleus (hammer), the incus (anvil), and the stapes (stirrup). They form a short bony chain spanning between the external ear and the middle ear. The malleus resembles a club, the incus a premolar tooth, and the stapes a stirrup. The malleus and the incus have a tightly fitting joint between them. The long process of the incus is bent near its lower end and carries a small bony knob that forms a loose joint with the head of the stapes. The stapes is about 3 m.m. high and weighs slightly less than 3 mgs. It lies almost horizontally at right angles to the long process of the incus. The footplate of the stapes fits neatly in the oval window, which is one of the two openings in the wall of the bony labyrinth, where it is held in place by the annular ligament.

When sound is transmitted through the ear canal, the malleus, which is attached to the eardrum, moves in and out with the movements of the drum membrane in response to the sound. The incus that is attached to the malleus moves with the malleus. The stapes that is coupled to the incus does not move in and out of the oval window, but rocks about the lower pole of its footplate as it transmits the vibrations to the inner ear. The footplate of the stapes fits neatly into the oval window, thereby concentrating the sound in a small area.

A disease of the middle ear known as fixation of the stirrup by otosclerosis causes progressive hearing loss in early and middle adult life. Otosclerosis is the abnormal formation of spongy bone in the inner ear that causes the stapes to become immobilized. In its early and actively expanding stage the nodule of the softened bone becomes large enough to reach the oval window containing the footplate of the stirrup. Increasing pressure caused by expanding nodule begins to impede the vibratory movements of the stapes in response to sound striking the drum membrane.

When the stapes becomes diseased, it may be removed and a stapedial prosthesis may be implanted in its place, all by well-known surgical techniques. One of the most widely-used stapedial prostheses is a piston-like member formed of Titanium, stainless steel, “Teflon” (a registered trademark of DuPont for a tetrafluoroethylene-hexa-florapropylene copolymer), polyethylene or other biocompatible material. Such prostheses are illustrated in U.S. Pat. Nos. 3,196,462; 3,711,869; 3,931,684; 4,292,693 and 5,171,240 and in the publication Catalog, Micro-Surgery Instruments and Implants, Richard Manufacturing Co., 1965.

Fixation of the stirrup bone can be corrected surgically by an operation known as stapedectomy. This operation involves the removal of the affixed stirrup bone and replacement by a plastic or narrow substitute, such as TEFLON (registered trademark) piston. The operation restores the vibration characteristics of the chain of tiny bones of the middle ear.

The operation is extremely delicate and difficult since the middle ear bones are the smallest bones of the body. The surgical implantation of a replacement stapes generally uses an instrument known as cupforceps or alligator forceps to hold the prosthetic piston as shown in FIG. 4. The cupforceps or alligator forceps are held in a similar fashion like holding a pair of scissors. This is inherently unstable, since the surgeon is applying closure pressure on the instrument while, at the same time trying to position the prosthetic piston in place.

This typically causes vibration at the tip of the instrument if the surgeon is inexperienced. Another factor contributing to the difficulty of the operation is that the operation is carried out through the ear canal by means of an ear speculum and a microscope.

Because the tip of the cupforceps is typically thick in relation to the prosthetic piston, and because of the narrow field of the operation, it is difficult to see the prosthetic piston tip for accurate implantation into the top of the footplate and for attaching to the lenticular process of the incus. This difficulty is highlighted in FIG. 4, which is a view into the ear canal through the ear speculum.

The incus bone is very thin and sensitive to any pressure and breakdown. Securing and closing the open ring over the fragile incus by mechanical cramping can be very complicated. Incus bone damage or even pressure on the bone can easily cause irreversible damage and may result in complete deafness.

There is therefore a need for a new and improved stapedial prosthesis and an instrument for use in implanting a prosthetic piston in the middle ear in a stapedectomy procedure that will resolve the above-mentioned difficulties.

### 3. OBJECTS AND SUMMARY OF THE INVENTION

It is therefore an object of the present invention to provide a novel improved stapedial prosthesis and an instrument that simplifies the implantation of a prosthetic piston in a stapedectomy procedure such as the procedure described in Hanwong; (U.S. Pat. No. 5,171,240.). The improvement essentially consists of a slotted ball or disc and nail porter securement stapedial piston. It is understood that if the slotted ball is exemplified then a disc may be used instead of the slotted ball.

It is another object of the present invention to provide an improved stapedial prosthesis and an instrument that does not obstruct the surgeon's narrow field of view when implanting a prosthetic piston in a stapedectomy procedure.

It is still another object of the present invention to provide an improved stapedial prosthesis and an instrument that is relatively much easier to use than cupforceps or alligator forceps, which are typically used in stapedectomy procedures.

It is yet another object of the present invention to provide an improved stapedial prosthesis and an instrument that minimizes the vibration at the tip of the instrument during implantation in a stapedectomy procedure due to the surgeon's inexperience or inherent difficulties associated with using cupforceps or alligator forceps.

It is still further another object of the present invention to provide an improved stapedial prosthesis and an instrument that enables even an inexperienced surgeon to accurately implant a prosthetic piston in a stapedectomy procedure.

It is another object of the present invention to provide an improved stapedial prosthesis and an instrument that is comfortable to handle thereby minimizing vibrations at the tip of the instrument and to provide relatively greater ease in implanting the prosthesis in a stapedectomy procedure.

It is yet another object of the present invention to provide an improved stapedial prosthesis to eliminate pressure on the lenticular process of the incus when the nail porter is in place thereon and eliminates any breakdown of the incus.

It is still further another object of the present invention to provide an improved stapedial prosthesis which closes and opens the nail porter without causing damage to the incus.

In summary, the present invention provides an improved stapedial prosthesis and an instrument for implantation of a prosthetic piston in a stapedectomy procedure that makes the said procedure more facile.

### BRIEF DESCRIPTION OF THE DRAWINGS

FIG. 1 is a diagram illustrating the sound-transmitting mechanism of a human ear.

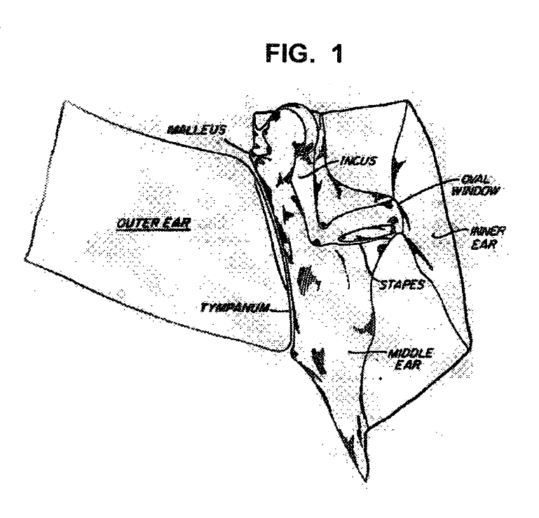

FIG. 2 is a similar to FIG. 1 with the stapes removed and an embodiment of the present invention, the improved stapedial prosthesis, substituted therefore.

FIG. 3 is a view of the slotted ball piston of the present invention, including nail porter with the head and tail of the nail.

FIG. 3A is a view of the slotted disc piston of the present invention, including nail porter with the head and tail of the nail.

FIG. 4 is a view looking into the ear canal with an ear speculum during a stapedectomy procedure using prior art cupforceps.

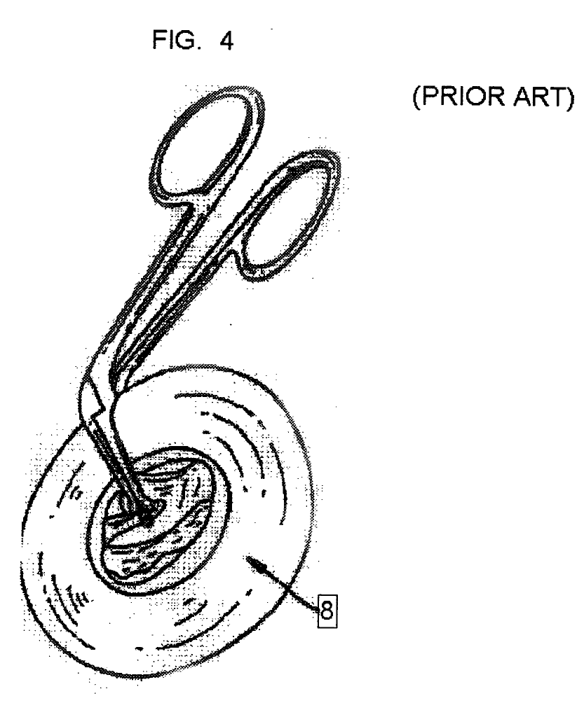

FIG. 5 is a perspective view of the instrument according to the present invention.

FIG. 6 is a perspective view of the instrument according to the present invention as shown in FIG. 5, including the scissors handle.

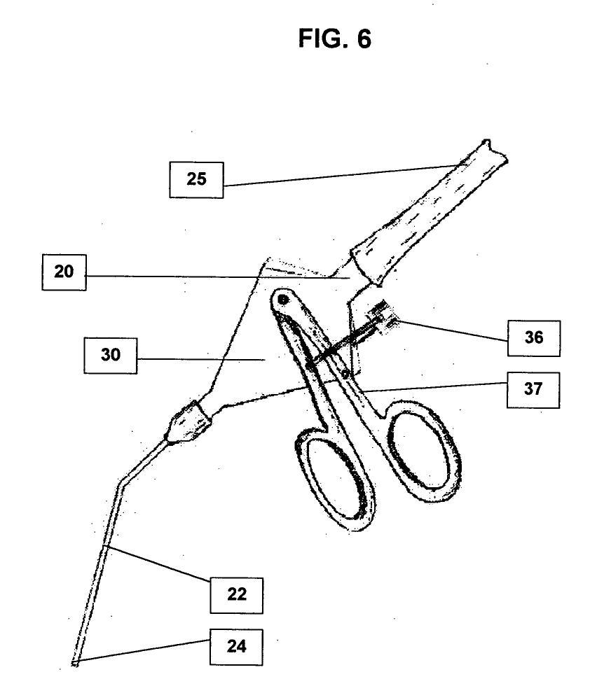

FIG. 7 is an enlarged sectional side elevational view of the adapter handle of the instrument as shown in FIG. 5.

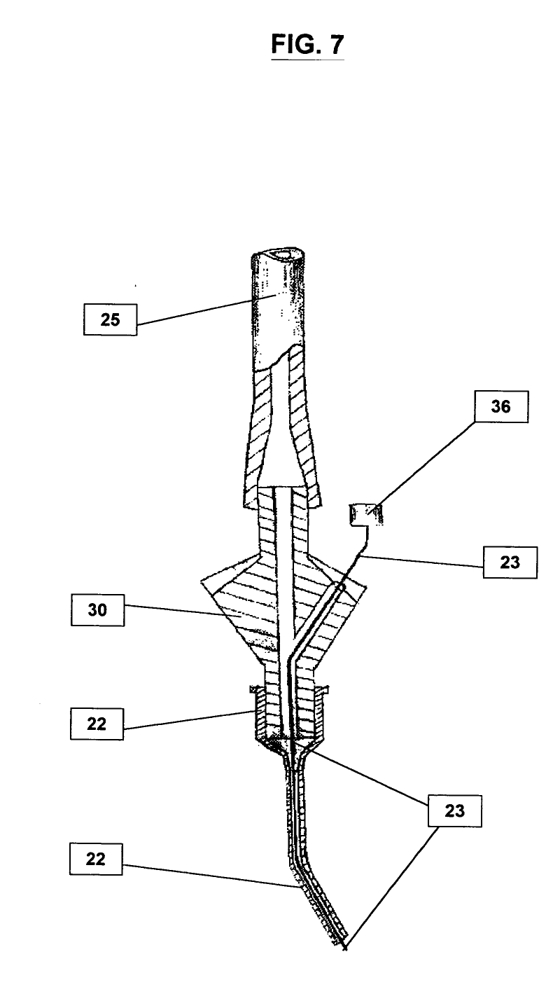

FIG. 8 is an enlarged side elevational view (A) and frontal view (B) of the tip of the instrument as shown in FIG. 5 showing the prosthetic nail in the tube end, secured thereby.

FIG. 9 is an enlarged, fragmentary, perspective view of the middle ear, with portions shown in cross-section and including an enlarged fragmentary view of the tip of the instrument as shown in FIG. 5 displaying a prosthetic piston secured thereby.

FIG. 10 is an enlarged, longitudinal cross-sectional view through the ear canal, showing a fragmentary view of the instrument with a prosthetic piston secured thereto while being positioned into the middle ear.

FIG. 11 is a view looking down into the ear canal, showing the prosthetic piston held by the tip of the instrument as shown in FIG. 8 and secured to the incus and fitted into a footplate hole of a previously removed stapes in the oval window.

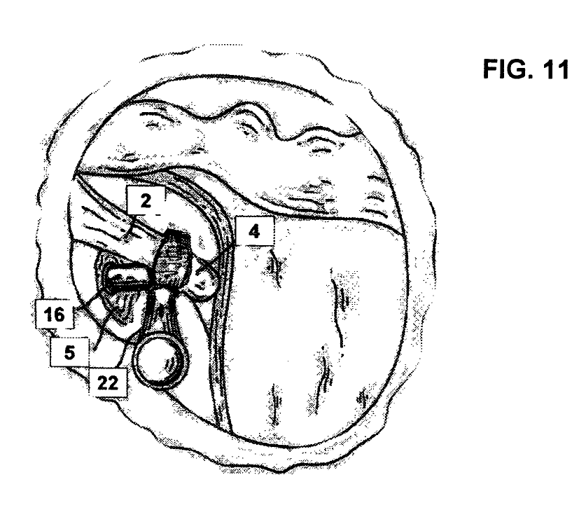

Other features and advantages of the present invention will become apparent as the following detailed description is taken together with the accompanying drawings.

### 5. DETAILED DESCRIPTION OF THE INVENTION

The numerations of the figures referring to the drawings are as follows:

The middle ear includes:

2-Incus, 4-Incus head (Lenticular process), 5-Oval window, 8-Ear canal,

The prosthesis implant includes:

12-Slotted ball or disc, 14-Annular opening of slotted ball or disc, 16-Piston, 17-Tip of Nail, 18-Nail Porter, 19-Nail head,

The inserter instrument includes:

20-Inserter handle, 22-Tube, 24-Tip of Tube, 25-Vacuum pipe, 23-Narrow rod , 30-Adapter, 36-Push button, 37-Scissors handle,

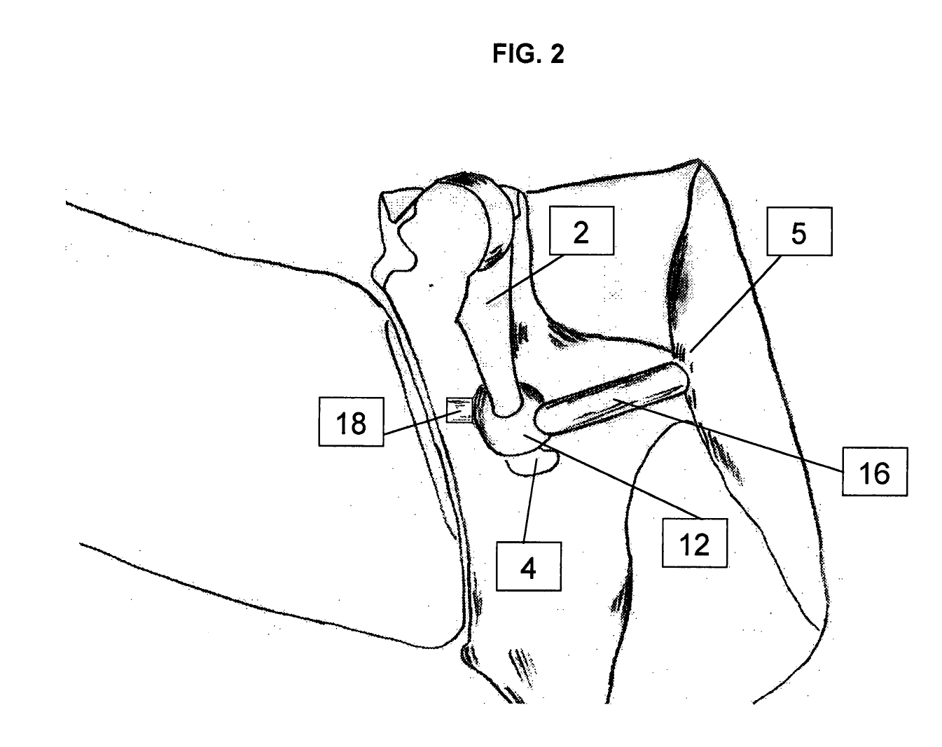

Referring to the drawings, particularly to FIG. 2, there is shown a stapedial prosthesis of the present invention surgically implanted in the middle ear after removal of the stapes.

The prosthesis includes a slotted ball or disc portion 12 having received therein a portion of the lenticular process 4 of the incus and a piston rod portion 16 whose free end engages the oval window 5. Pivotally mounted on the slotted ball or disc portion 12 is a narrow nail porter 18 which, after positioning the prosthesis, is pushed to the position shown in loose engagement with the lenticular process 4 of the incus.

The prosthesis implanted in this manner transmits sound vibrations from the incus to the oval window 5 of the middle ear.

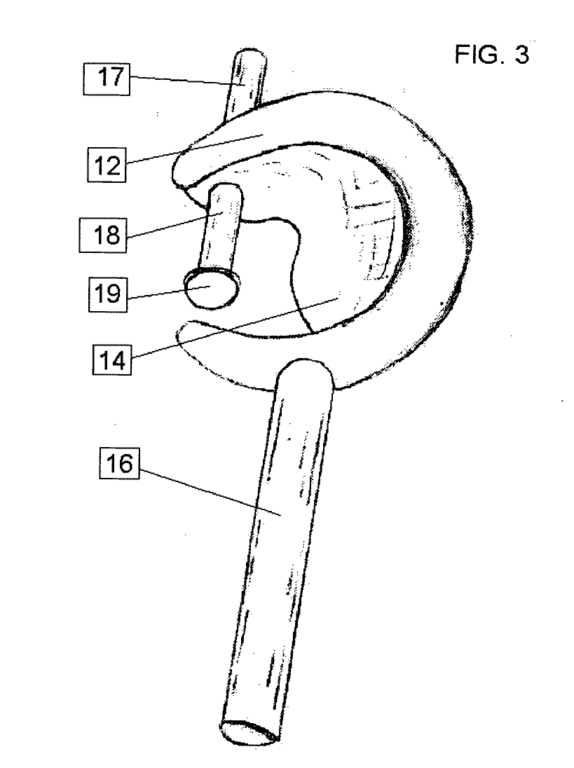

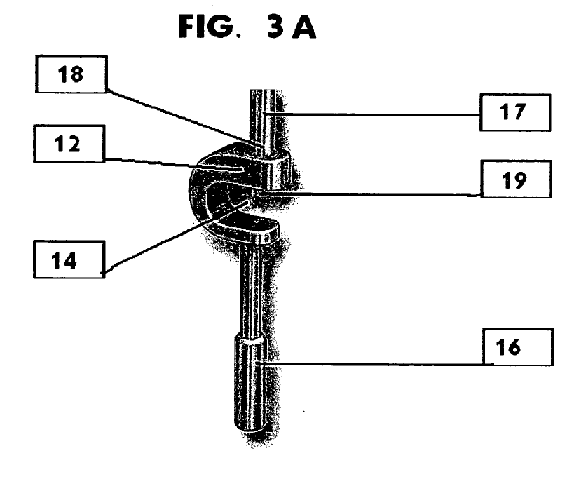

The prosthesis will now be described in greater detail by reference to FIG. 3 which shows the piston portion of the prosthesis .The slotted ball or disc portion 12 and piston rod portion 16 which is in the center of the slotted ball or disc as one piece, comprise the prosthesis. These parts generally are integrally formed from a single piece of stainless steel, Titanium ,Teflon or other biocompatible materials, used to advantage in stapes replacement surgery. The slotted ball or disc portion 12 is incorporated with the longitudinal axis of the rod piston portion 16, that rest on the top surface of the lenticular process of the incus 4 after implantation has provided more tolerance in fitting the slotted ball or disc portion 12 over the end of the lenticular process of the incus 4 and thereby minimizing the risk of pressure necrosis.

In typical dimensions:

The rod piston portion 16 may range between 2.0 mm to 10.0 mm, preferably between 3.5 mm to 6.0 mm in length and between 0.2 mm and 1.5 mm, preferably 0.4 mm and 0.8 mm in diameter.

The outside diameter of the ball or disc portion 12 may range between 1.0 mm to 5.0 mm preferably 1.4 mm to 2.5 mm.

The diameter of the annular opening of slotted ball or disc portion 14 may range between 0.4 mm to 2.0 mm preferably 0.6 mm to 1.2 mm.

The diameter of nail porter 18 is about 0.1 mm to 0.6 mm.

The length of nail porter 18 is about 1.4 mm to 6.0 mm.

The diameter of nail tip portion 17 is about 0.1 mm to 1.0 mm.

The diameter of nail head portion 19 is about 0.2 mm to 1.3 mm.

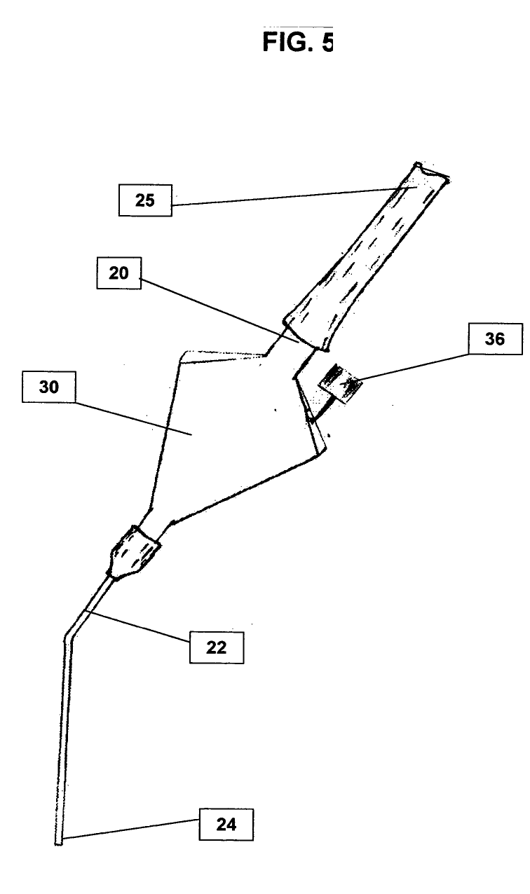

The instrument shown in FIG. 5, includes an inserter handle 20, an adapter 30, and tube 22 secured wholly or with each other.

The instrument is preferably made from stainless steel but other suitable materials may also be used in the medical field. The inserter handle 20 includes a connection to the vacuum source pipe 25, to provide a vacuum to hold the prosthetic piston secured thereby.

Tube 22 is used as a suction tube straight or bent at an obtuse angle. The tube 22 is long enough to allow its insertion into the ear canal and then continues into the middle ear. The obtuse angle of the tube thereby allows the surgeon's hand to be offset to the side of the ear canal opening thus permitting the surgeon an unobstructed view into the ear canal when using the instrument.

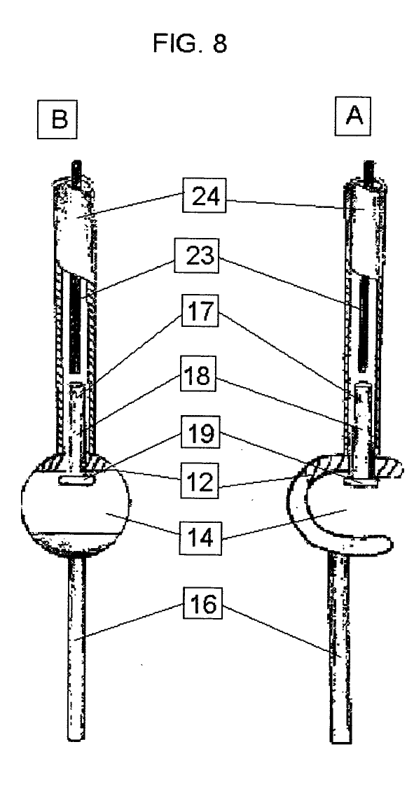

The free end of the tip of the tube 24 is shown in FIG. 8.

A prosthetic implant, preferable made of stainless steel, titanium or plastic such as, for example, TEFLON (registered trademark) or other suitable materials is positioned in the free end of the tube as shown in FIG. 8. The vacuum force prevents the prosthesis from falling, rotating or tilting from side to side in the interior of the tube. The tip of the tube provides a break for limiting the rotation of the prosthetic implant in an axis parallel to the said prosthetic implant, as shown in FIG. 8. The annular opening of the slotted ball or disc 14 of the prosthetic implant facilitates installation of the prosthesis as shown in FIGS. 8 to 10. Inside the tube there is a narrow rod 23 that pushes the nail porter 18 to its final position and the prosthetic implant is now at its desired place.

### Operation

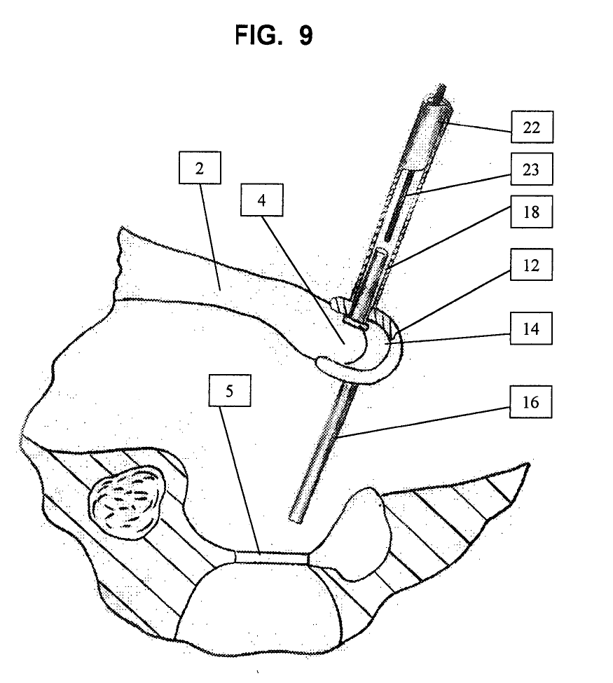

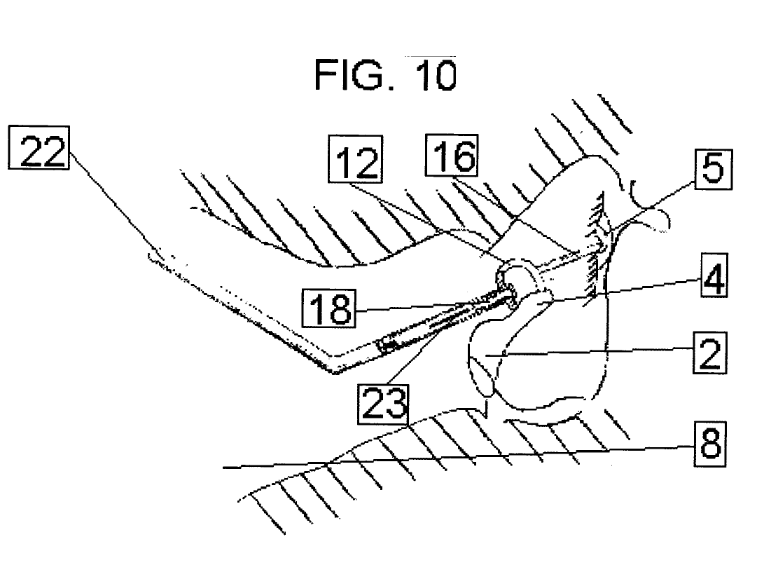

The prosthetic implant as shown in FIG. 3 includes a piston portion 16 and the slotted ball or disc 12 with a nail porter 18. The prosthetic implant is secured to the free end of the tip of the vacuum tube 24 as shown in FIG. 8 by positioning the nail porter 18 in the interior end of the tube thus assisting in retaining the prosthesis in the open end of the tube 24 while it is being positioned in the middle ear as shown in FIG. 10. The stapes in the middle ear are surgically removed in a conventional manner. A small hole is then made on the remaining footplate of the previously removed stapes in a conventional manner. The hole is adapted to the size of the prosthetic piston 16. The footplate of the stapes may also be removed if desired by using the so-called large hole technique. An annular opening 14 is the result of the cutting of slots in the ball or disc 12 of the prosthetic implant as shown in FIG. 3. The annular opening 14 is adapted to enable the slotted ball or disc 12 to slip over the lenticular process of the incus 4, as shown in FIG. 9. The instrument comprising the prosthetic implant is then inserted into the external ear canal 8 as shown in FIG. 10 and continues into the middle ear until the prosthetic implant 10 is positioned into the small hole of the oval window 5, as shown in FIGS. 9 and 10. The slotted ball or disc 12 is then pressed into the inwardly curving portion of the lenticular process of the incus 4. The prosthetic implant is installed at the posterior of the lenticular process of the incus 4, as shown in FIGS. 9. and 10.

After the prosthetic implant is securely in place in the small hole of the oval window 5 and over the lenticular process of the incus 4, the instrument is retrieved by pushing the narrow rod 23 in the interior end of the tube 24, about 2 mm in length. The narrow rod disengages the nail porter 18 as it locks the slotted ball or disc 12. Finally, the instrument is carefully removed from the ear canal.

To remove the prosthetic implant from the lenticular process of the incus 4, the tip of the tube 24 is secured to the tip of the nail porter 17 at the slotted ball or disc 12 as described above. The instrument is slowly withdrawn by means of a vacuum and the nail porter 18 in the free end of the tube 24 so that the lock is opened and the prosthetic implant can be disengaged from the lenticular process of the incus 4 through the annular opening 14 of the slotted ball or disc 12. as shown in FIG. 9.

The prosthetic implant is then removed from the ear canal. By using the instrument of the invention the surgeon has a complete view of the tip of the instrument during the whole procedure as shown in FIG. 10.

While this invention has been described as having the preferred design, it is understood that it is capable of further modifications, uses and/or adaptations of the invention following in general the principle of the invention and including such departures from the present disclosure as come within known or customary practice in the art to which the invention pertains and as may be applied to the essential features set forth and fall within the scope of the invention or the limits of the appended claims.

What is claimed is:

1. In a prosthesis for replacing the stapes of the middle ear, said prosthesis being of the piston type comprising a slotted ball or disc for receiving the lenticular process of the incus and a nail porter mounted to a pivot about the open end of said said slotted ball or disc, the improvement comprising:
a means mounted on said slotted ball or disc for permitting locking the incus in the slot by a porter nail.

2. An instrument for holding the prosthesis as claimed in claim 1 for use in a medical procedure, comprising:
i) an inserter handle;
ii) an adapter connecting the inserter handle and the tube,
iii) a tube attached to said handle;
iv) said handle connected to a vacuum system,
v) said tip of the tube for holding part of the prosthesis therein thereby securing said prosthesis for implantation and removal.
vi) a narrow rod inserted in the tube.

3. An instrument as claimed in claim 2, wherein: said tube can also include a bend at an obtuse angle and the top opening of the tube is directed away from the direction of the bend.

4. An instrument as claimed in claim 2 for holding and positioning a prosthesis within the ear canal in a stapedectomy procedure, said instrument comprising a narrow rod and a break for limiting the rod pushing of the prosthesis.

5. The inserter handle portion of claim 2 is secured to a tube which contains a narrow rod.

6. The inserter handle portion of claim 5 wherein said handle is connected to a vacuum source.

7. An instrument of claim 2 wherein the tube, inserter handle and adapter is secured wholly or partly with each other.

8. An instrument of claim 2 wherein the tube, inserter handle and adapter is welded wholly or partly with each other.

9. An instrument of claim 2 wherein the tube, inserter handle and adapter is swaged wholly or partly with each other
